SEQUENCE LISTING (9 sequences)

SEQ ID NO: 1 - protein, 1465 aa, Homo sapiens [sequence omitted]

SEQ ID NO: 2 - protein, 477 aa, Homo sapiens
MRGLGTCLATLAGLLLTAAGETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKEPPVPIAPPQLASVGATYLWIQLNANSINGDGPIVAREVEYCTASGSWNDRQPVDSTSYKIGHLDPDTEYEISVLLTRPGEGGTGSPGPALRTRTKCADPMRGPRKLEVVEVKSRQITIRWEPFGYNVTRCHSYNLTVHYCYQVGGQEQVREEVSWDTENSHPQHTITNLSPYTNVSVKLILMNPEGRKESQELIVQT

SEQ ID NO: 3 - protein, 280 aa, Homo sapiens
MRGLGTCLATLAGLLLTAAGETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKE

SEQ ID NO: 4 - protein, 20 aa, Artificial Sequence
  Synthetic peptide
ETFSGGCLFDEPYSTCGYSQ

SEQ ID NO: 5 - protein, 21 aa, Artificial Sequence
  Synthetic peptide
EGEGDDFNWEQVNTLTKPTSD

SEQ ID NO: 6 - protein, 19 aa, Artificial Sequence
  Synthetic peptide
TPHFLRIQNVEVNAGQFAT

SEQ ID NO: 7 - protein, 18 aa, Artificial Sequence
  Synthetic peptide
GIDVRDAPLKEIKVTSSR

SEQ ID NO: 8 - protein, 21 aa, Artificial Sequence
  Synthetic Construct
CGEGDDFNWEQVNTLTKPTSD

SEQ ID NO: 9 - protein, 20 aa, Artificial Sequence
  Synthetic Construct
GTQDETGNFDWPVSEDLNKT

(19) United States
(12) Patent Application Publication
(10) Pub. No.: US 2021/0100920 A1
(43) Pub. Date: Apr. 8, 2021

(54) METHODS AND AGENTS FOR THE DETECTION AND TREATMENT OF CANCER

(71) Applicant: CASE WESTERN RESERVE UNIVERSITY, Cleveland, OH (US)

(72) Inventors: Susann Brady-Kalnay, Cleveland, OH (US); Mette L. Johansen, Cleveland, OH (US)

(21) Appl. No.: 16/608,021

(22) PCT Filed: May 24, 2017
(86) PCT No.: PCT/US2018/029179

§ 371 Date: Oct. 24, 2019

Related U.S. Application Data
(60) Provisional application No. 62/489,231, filed on Apr. 24, 2017.

(51) Int. Cl. A61K 49/10; A61K 49/00; A61K 49/08; A61K 49/14

(52) U.S. Cl. CPC A61K 49/108; A61K 49/14; A61K 49/085; A61K 49/0002

(57) ABSTRACT

An agent for use in detecting, monitoring, and/or imaging cancer cells and/or cancer cell metastasis, migration, dispersal, and/or invasion, and/or for treating cancer in a subject includes a targeting peptide and at least one of a detectable moiety, therapeutic agent, or a theranostic agent that is directly or indirectly linked to the targeting peptide. The targeting peptide specifically binds to and/or complexes with a proteolytically cleaved extracellular fragment of an immunoglobulin (Ig) superfamily cell adhesion molecule that is expressed by a cancer cell or another cell in the cancer cell microenvironment.

### RELATED APPLICATION

This application claims priority of U.S. Provisional Application Ser. No. 62/489,231 filed Apr. 24, 2017, the subject matter of which is incorporated herein by reference in its entirety.

### BACKGROUND

Cancer detection and treatment are hindered by the inability to differentiate between cancer cells and normal cells. Better detection tools for cancer or tumor imaging are needed for earlier diagnosis of cancers. Molecular recognition of tumor cells would facilitate guided surgical resection. In order to improve surgical resection, targeted imaging tools must specifically label tumor cells, not only in the main tumor but also along the edge of the tumor and in the small tumor cell clusters that disperse throughout the body.

Targeted imaging tools designed to label molecules that accumulate in the tumor microenvironment may also be advantageous as therapeutic targeting agents, as they can identify both the main tumor cell population and areas with infiltrating cells that contribute to tumor recurrence. The ability to directly target the tumor cell and/or its microenvironment would increase both the specificity and sensitivity of current treatments, therefore reducing non-specific side effects of chemotherapeutics that affect cells throughout the body.

### SUMMARY

Embodiments described herein relate to agents and methods for use in detecting, monitoring, and/or imaging cancer cells and/or cancer cell metastasis, migration, dispersal, and/or invasion, and/or treating cancer in a subject in need thereof. The agent includes a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment, and at least one of a detectable moiety, therapeutic agent and/or a theranostic agent that is coupled to the targeting peptide. In some embodiments, the targeting peptide is coupled to the detectable moiety, therapeutic agent and/or a theranostic agent via a linking molecule.

In some embodiments, the Ig superfamily cell adhesion molecule can include a cell surface receptor protein tyrosine phosphatase (PTP) type IIb, such as PTPμ or a PTPμ like molecule.

In another embodiment, the extracellular fragment can have an amino acid sequence of SEQ ID NO: 2, and the targeting peptide can specifically bind to and/or complex with SEQ ID NO: 2. In a further embodiment, the targeting peptide can include a polypeptide having an amino acid sequence that has at least 80% sequence identity to about 10 to about 50 consecutive amino acids of SEQ ID NO: 3. Examples of polypeptides having an amino acid sequence with an at least about 80% sequence identity to SEQ ID NO: 3 can be polypeptides having an amino acid sequence selected from the group consisting of SEQ ID NO: 4, SEQ ID NO: 5, SEQ ID NO: 6, and SEQ ID NO: 7.

In some embodiments, the therapeutic agent and/or theranostic agent can include an anti-cancer agent.

In other embodiments, the detectable moiety can include a chelating agent and a single metal radiolabel. The chelating agent can include, for example, dodecanetetraacetic acid (DOTA). The single metal radiolabel can be selected from the group consisting of a gadolinium ion or a gallium ion.

In particular embodiments, the agent can be a molecular probe that has the following formula (I):

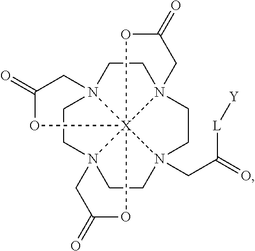

wherein X is selected from Ga or Gd metal ion, L is a linking molecule, and Y is a polypeptide having an amino acid sequence selected from the group consisting of SEQ ID NO: 4, SEQ ID NO: 5, SEQ ID NO: 6, SEQ ID NO: 7, and SEQ ID NO: 8 and pharmaceutically acceptable salts thereof.

In some embodiments, the linking molecule is not a contiguous portion of either the polypeptide or chelating agent and covalently joins an amino acid of the polypeptide to a carboxyl group of the chelating agent. In some embodiments, the linking molecule can include an amino acid residue. For example the linking molecule can include a lysine residue.

The cancer detected or treated with the agent can be of any type of cancer including, but not limited to, bone cancer, bladder cancer, brain cancer, neuroblastoma, breast cancer, cancer of the urinary tract, carcinoma, cervical cancer, astrocytoma, brain stem glioma, NCS atypical teratoid/rhabdoid tumor, CNS embryonal tumor, CNS Germ Cell tumors, craniopharyngioma, ependymoma, kidney tumors, acute lymphoblastic leukemia, acute myeloid leukemia, and other types of leukemia; Hodgkin lymphoma, non-Hodgkin lymphoma, Ewing sarcoma, osteosarcoma and malignant fibrous histiocytoma of the bone, rhabdomyosarcoma, soft tissue sarcoma, Wilms' tumor, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, stomach cancer, testicular cancer, thyroid cancer, and uterine cancer.

In some embodiments, the cancer cell can be, for example, a metastatic, migrating, dispersed, and/or invasive cancer cell, such as a metastatic, migrating, dispersed, and/or invasive brain cancer cell (e.g., glioma cell and, specifically, a glioblastoma multiforme (GBM) cell), lung cancer cell, breast cancer cell, prostate cancer cell, and/or melanoma.

The agent when used as a molecular probe can be detected in vivo by detecting, recognizing, or imaging the detectable moiety. The detectable moiety can be detected by at least one of magnetic resonance imaging, positron emission tomography (PET) imaging, computer tomography (CT) imaging, gamma imaging, near infrared imaging, or fluorescent imaging.

Other embodiments described herein also relate to methods of detecting, monitoring, and/or imaging cancer cells and/or cancer cell metastasis, migration, dispersal, and/or invasion in a subject. The method includes administering a molecular probe to the subject. The molecular probe includes a targeting peptide that specifically binds to and/or complexes with a proteolytically cleaved extracellular fragment of an immunoglobulin (Ig) superfamily cell adhesion molecule in the cancer cell microenvironment that is expressed by the cancer cell or an endothelial cell that supports survival of the cancer cell and a detectable moiety and/or theranostic agent that is coupled to the targeting peptide. The detectable moiety and/or theranostic agent can be directly coupled to the targeting peptide or indirectly coupled to the targeting peptide via a linking molecule or linker.

The molecular probe can be detected in vivo by detecting, recognizing, or imaging the detectable moiety. The detectable moiety can be detected by at least one of magnetic resonance imaging, positron emission tomography (PET) imaging, computer tomography (CT) imaging, gamma imaging, near infrared imaging, or fluorescent imaging.

Still other embodiments relate to a method of treating cancer in a subject in need thereof. The method can include administering to the subject a therapeutically effective amount of an agent that includes a targeting peptide that specifically binds to or complexes with a proteolytically cleaved extracellular fragment of an immunoglobulin (Ig) superfamily cell adhesion molecule (CAM) or its receptor that is expressed by a cancer cell or another cell in the cancer cell microenvironment and an anti-cancer agent that is coupled to the targeting peptide.

### BRIEF DESCRIPTION OF THE DRAWINGS

FIGS. 1A-B illustrate a structure and spectra of SBK2-Lys-(Gd-DOTA). (A) The macrocyclic chelator DOTA, attached to a lysine, was coupled to the N-terminal glycine of the PTPμ targeted peptide, SBK2, via an amide bond. The control agent, Scrambled-Lys-(Gd-DOTA), has the same structure but differs in the order of amino acids in the peptide. (B) MALDI-TOF spectra of the Scrambled-Lys-(Gd-DOTA) and SBK2-Lys-(Gd-DOTA) following complexation. The x axis is m/z and the y axis is intensity (a.u.).

FIG. 2 illustrates a graph showing SBK2-Lys-(Gd-DOTA) clears more slowly and at different rates compared to Scrambled-Lys-(Gd-DOTA). The clearance rate, calculated by the change in normalized T₁ value over time, of each agent administered at 0.2 mmol/kg in 10 minute increments are shown 10 minutes after injection of agent. The clearance rates for SBK2-Lys-(Gd-DOTA) at increments from 10 to 50 minutes are significantly lower than those of the Scrambled agent. While the Scrambled agent demonstrates a relatively constant clearance rate from 20 minutes-60 minutes, the rate of SBK2 clearance is initially negative, indicating that T₁ values continue to drop for up to 30 minutes, before starting to return to their initial values.

FIGS. 3A-B illustrate graphs showing SBK2-Lys-(Gd-DOTA) shows sustained labeling of LN-229 heterotopic glioma xenografts in a dose dependent manner (A) The rate of agent clearance was calculated from the change in T₁ relaxation time/time (min) in mice with LN-229 flank tumors administered the indicated doses of each agent and determined between 15 and 30 min following injection of the agent. Each bar represents a group of 4-8 flank tumor-bearing animals. The clearance rates for SBK2 and the control agent are statistically significant at all 4 doses (p<0.02). (B) Normalized T₁ relaxation times over time for mice administered the indicated agent at 0.1 mmol Gd/kg in LN-229 flank tumor (top) or a control area (bottom). The agents were injected following acquisition of 5 baseline scans. Upon injection (indicated by arrow), the normalized T₁ relaxation times decrease at a similar rate and to a similar extent, and the specific SBK2 agent shows a sustained decrease in normalized relaxation time while the Scrambled control T₁ times increase more rapidly. The lines show the mean+/−SE of 4 mice treated with SBK2 agent, and 5 mice treated with Scrambled agent. T₁ times obtained from tumor ROIs were significantly different from 25 min to 62.5 min post injection (p<0.05 at all time points: p=0.04 at 25 min, p=0.02 at 27.5 min, and p<0.01 for all other time points). No statistically significant differences in T₁ times for the control tissue ROIs were observed.

FIG. 4 illustrates images showing LN-229 flank tumors in mice treated with SBK2-Lys-(Gd-DOTA) retain Gd longer than those in mice treated with the control agent. Representative Gd concentration maps of flank tumors overlaid onto T₁-weighted images in animals treated with 0.1 mmol Gd/kg of either the non-specific Scrambled agent (right) or SBK2 agent (left) at baseline (time 0), and at 15, 30 and 60 min following injection of the agent. The SBK2-Lys-(Gd-DOTA) concentration is sustained longer than the non-specific agent. The color bar indicates Gd concentration (mM) with blue representing no Gd and red representing 0.06 mM Gd.

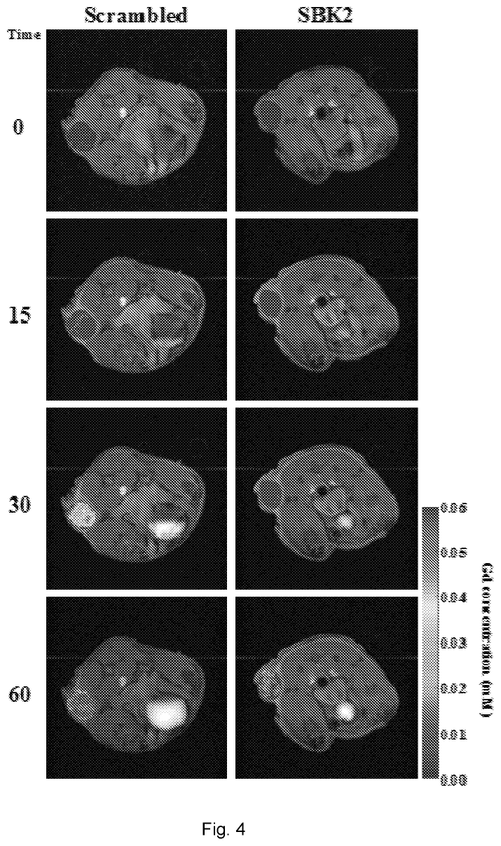

FIGS. 5A-B illustrate graphs showing SBK2-Lys-(Gd-DOTA) shows prolonged labeling of intracranial CNS-1 tumors at 0.2 mmol Gd/kg compared to Scrambled-Lys-(Gd-DOTA). (A) The rate of agent clearance from 7d CNS-1 tumors was determined by the change in T₁ map value/time (min) from 20 to 35 min following intravenous injection of agent and is significantly different (p=0.026) between the mice treated with SBK2 (SEQ ID NO:2) and Scrambled agent. N=4 for each agent. (B) Normalized T₁ map values over time for mice administered the indicated agent at 0.2 mmol Gd/kg in CNS-1 intracranial tumor (top) or a contralateral control area (bottom). The agents were injected following acquisition of 5 baseline scans. Upon injection, the T₁ map values decrease at a similar rate and to a similar extent but the specific SBK2 agent is cleared more slowly than the non-specific Scrambled agent in tumor. The lines show the mean+/−SE of 4 mice treated with SBK2 agent, and 4 mice treated with Scrambled agent. SB K2 and Scrambled agent but show the same trend observed with flank tumors. The decrease in T₁ signal is less pronounced in the brains compared to the flanks, despite twice the dose. No statistically significant differences in T₁ map values for the control tissue ROIs were observed.

FIG. 6 illustrates images showing orthotopic intracranial CNS-1 tumors in mice treated with SBK2-Lys-(Gd-DOTA) retain Gd longer than those in mice treated with the control agent. High resolution T₂-weighted images for representative animals treated with 0.2 mmol Gd/kg of either Scrambled-Lys-(Gd-DOTA) (left) or SBK2-Lys-(Gd-DOTA) (right) are shown at the top. Heat maps depicting average post-contrast Gd concentration throughout the brain are superimposed on T₁-weighted images. T₁ mapping scans from the time of agent injection out to 30 min were averaged together and the Gd concentration was determined on a pixel-by-pixel basis. The color bar indicates Gd concentration (mM) with blue representing no Gd and red representing 0.030 mM Gd.

FIG. 7 illustrates an image and graph showing sustained PET imaging over time of flank tumors in mice using a PTPμ-DOTA-Gallium molecular probe.

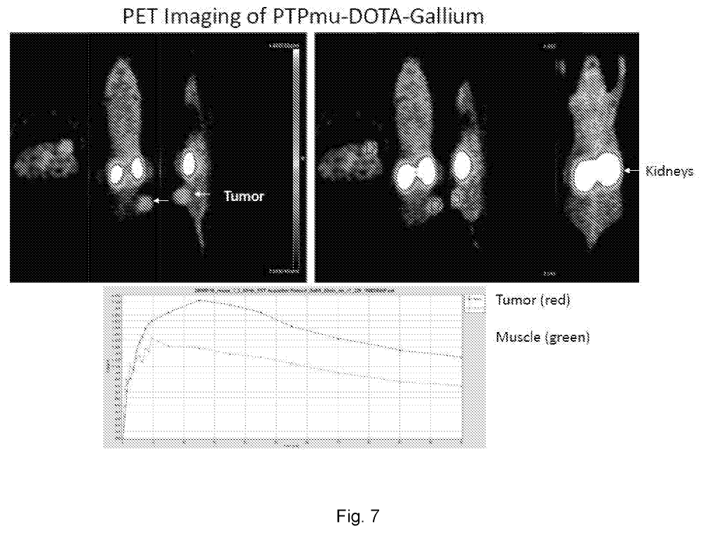

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The term “agent” is used herein to denote a chemical compound, a mixture of chemical compounds, a biological macromolecule, or an extract made from biological materials.

The terms “antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

The term “epitope” includes any protein determinant capable of specific binding to an immunoglobulin. Epitope determinants usually consist of chemically active surface groupings of molecules such as amino acids or sugar side chains and usually have specific three-dimensional structural characteristics, as well as specific charge characteristics.

The term “gene” or “recombinant gene” refers to a nucleic acid comprising an open reading frame encoding a polypeptide, including both exon and (optionally) intron sequences.

The term “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “monoclonal” refers to an antibody that specifically binds to a sequence of amino acid and/or a specific epitope of an antigen.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to agents for use in detecting, monitoring, and/or imaging cancer cells and/or cancer cell metastasis, migration, dispersal, and/or invasion in a subject, methods of detecting, monitoring, and/or imaging cancer cells and/or cancer cell metastasis, migration, dispersal, and/or invasion in a subject, methods of determining and/or monitoring the efficacy of a cancer therapeutic and/or cancer therapy administered to a subject in need thereof, and methods of treating a cancer in a subject in need thereof.

The agents described herein include a targeting peptide that specifically binds to and/or complexes with a proteolytically cleaved extracellular fragment of an immunoglobulin (Ig) superfamily cell adhesion molecule in the cancer cell microenvironment that is expressed by the cancer cell or an endothelial cell, which supports survival of the cancer cell, and at least one of a detectable moiety, therapeutic agent, or a theranostic agent that is directly or indirectly linked to the targeting peptide. The Ig superfamily cell adhesion molecule can include an extracellular homophilic binding portion, which can bind in homophilic fashion or engage in homophilic binding in a subject.

The agents can be administered systemically to a subject and readily target cancer cells associated with proteolytically cleaved extracellular fragment of the immunoglobulin (Ig) superfamily cell adhesion molecule, such as metastatic, migrating, dispersed, and/or invasive cancer cells. In some embodiments, the agent after systemic administration can cross the blood brain barrier to define cancer cell location, distribution, metastases, dispersions, migrations, and/or invasion as well as tumor cell margins in the subject. In other embodiments, the agent after systemic administration can inhibit and/or reduce cancer cell survival, proliferation, and migration.

It was found that targeting peptides, which can specifically bind to and/or complex with these proteolytically cleaved extracellular fragments or segments, can be used to target detectable moieties, therapeutic agents, and/or theranostic agents to cancer cells as well as cancer cell metastasis, migrations, dispersals, and/or invasions in a subject. When the agents are used as molecular probes, which include a targeting peptide coupled or linked to the detectable moiety, the molecular probes were shown to clearly demarcate the tumor cells in tissue sections and tumor “edge” samples, suggesting that the molecular probe can be used as diagnostic tools for molecular imaging of metastatic, dispersive, migrating, or invading cancers or the tumor margin. Systemic introduction of molecular probe as described herein resulted in rapid and specific labeling of the flank tumors and intracranial tumors within minutes. Labeling occurred primarily within the tumor, however a gradient of molecular probe at the tumor margin was also observed. There is also a signal amplification effect as extracellular fragments accumulate over time.

The agents described herein can therefore be used in a method of detecting cancer cells and/or cancer cell metastasis, migration, dispersal, and/or invasion as well as in a method of treating cancer in a subject in need thereof. The methods can include administering to a subject a an agent that includes a targeting peptide that binds to and/or complexes with the proteolytically cleaved extracellular fragment of the Ig superfamily cell adhesion molecule in the cancer cell or tumor cell microenvironment and detecting the molecular probe bound to and/or complexed with the proteolytically cleaved extracellular fragment of the Ig superfamily cell adhesion molecule in the cancer cell or tumor cell microenvironment.

In one example, the Ig superfamily cell adhesion molecule includes RPTP type IIb cell adhesion molecules. In another example, Ig superfamily cell adhesion molecules can include RPTPs of the PTPμ-like subfamily, such as PTPμ, PTPK, PTPp, and PCP-2 (also called PTPλ). PTPμ-like RPTPs include a MAM (Meprin/A5-protein/PTPμ) domain, an Ig domain, and FNIII repeats. PTPμ can have the amino acid sequence of SEQ ID NO: 1, which is identified by Genbank Accession No. AAI51843.1. It will be appreciated that the PTPμ gene can generate splice variants such that the amino acid sequence of PTPμ can differ from SEQ ID NO: 1. In some embodiments, PTPμ can have an amino acid sequence identified by Genbank Accession No. AAH51651.1 and Genbank Accession No. AAH40543.1.

Cancer cells and/or endothelial cells, which support cancer cell survival, that express an Ig superfamily cell adhesion molecule and that can be proteolytically cleaved to produce a detectable extracellular fragment can include, for example, cancer cells and/or other cells in the tumor microenvironment, such as stem cells, endothelial cells, stromal cells and immune cells that promote their survival.

The cancers detected and/or treated by the agents described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America)

The agents can also be used to detect and/or treat a variety of cancers or other abnormal proliferative diseases, including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoictic tumors of myeloid lineage, including acute and chronic myelogenous leukemias and promyclocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are detected, treated, or prevented in the skin, lung, colon, rectum, breast, prostate, bladder, kidney, pancreas, ovary, or uterus. In other specific embodiments, sarcoma, melanoma, or leukemia is detected and/or treated.

In still other embodiments, the cancer cells that are detected and/or treated can include glioma cells, lung cancer cells, breast cancer cells, prostate cancer cells, and melanoma cells, such as invasive, dispersive, motile or metastic cancer cells can include glioma cells, lung cancer cells, breast cancer cells, prostate cancer cells, and melanoma cells. It will be be appreciated that other cancer cells and/or endothelial cells, which support cancer cell survival, that express an Ig superfamily cell adhesion molecule and that can be proteolytically cleaved to produce a detectable extracellular fragment can identified or determined by, for example, using immunoassays that detect the Ig superfamily cell adhesion molecule expressed by the cancer cells or endothelial cells.

In some embodiments, the targeting peptide (or targeting polypeptide) can include a polypeptide (or targeting polypeptide) that binds to and/or complexes with the proteolytically cleaved extracellular fragment of the Ig superfamily cell adhesion molecule. The targeting peptide can include, consist essentially of, or consist of about 10 to about 50 amino acids and have an amino acid sequence that is substantially homologous to about 10 to about 50 consecutive amino acids of a homophilic binding portion or domain of the proteleolytically cleaved extracellular fragment of the Ig superfamily cell adhesion molecule. By substantially homologous, it is meant the targeting polypeptide has at least about 80%, about 90%, about 95%, about 96%, about 97%, about 98%, about 99% or about 100% sequence identity with a portion of the amino acid sequence of the binding portion of the proteleolytically cleaved extracellular fragment of the Ig superfamily cell adhesion molecule.

In one example, the homophilic binding portion of the Ig superfamily cell adhesion molecule can include, for example, the Ig domain of the cell adhesion molecule. In another example, where the Ig superfamily cell adhesion molecule is PTPμ, the homophilic binding portion can include the Ig binding domain and the MAM domain.

In another aspect, the targeting peptide can have an amino acid sequence that is substantially homologous to about 10 to about 50 consecutive amino acids of the Ig binding domain and/or MAM domain of PTPμ (e.g., SEQ ID NO: 1) and readily cross the blood brain barrier when systemically administered to a subject. The development of the PTPμ targeting peptides can be based on a large body of structural and functional data. The sites required for PTPμ-mediated homophilic adhesion have been well characterized. In addition, the crystal structure of PTPμ can provide information regarding which regions of each functional domain are likely to be exposed to the outside environment and therefore available for homophilic binding and thus detection by a peptide.

In some embodiments, the proteolytically cleaved extracellular fragment of PTPμ(e.g., SEQ ID NO: 1) can include an amino acid sequence of SEQ ID NO: 2, the Ig and MAM binding region can comprise the amino acid sequence of SEQ ID NO: 3, and the polypeptide can have an amino acid sequence that is substantially homologous to about 10 to about 50 consecutive amino acids of SEQ ID NO: 2 or SEQ ID NO: 3. Examples of polypeptides that can specifically bind SEQ ID NO: 2 or SEQ ID NO: 3 and have an amino acid sequence that is substantially homologous to about 10 to about 50 consecutive amino acids of SEQ ID NO: 2 or SEQ ID NO: 3 are polypeptides that comprising an amino acid sequence selected from the group consisting of SEQ ID NO: 4, SEQ ID NO: 5 (SBK2), SEQ ID NO: 6, and SEQ ID NO: 7. Polypeptides comprising SEQ ID NO: 4, 5, 6, or 7 can recognize or bind to the MAM, Ig domain, or the FNIII repeats. In particular embodiments, the targeting peptide is a SBK2 polypeptide comprising an amino acid sequence SEQ ID NO:5.

In other embodiments, a polypeptide that binds to and/or complexes with the proteolytically cleaved extracellular fragment of the Ig superfamily CAM or its receptor that is expressed by a cancer cell or another cell in the cancer cell microenvironment can have an amino acid sequence of SEQ ID NO: 8. SEQ ID NO: 8 is substantially homologous to a portion of SEQ SEQ ID NO: 1 or SEQ ID NO: 2 and can specifically bind to SEQ ID NO: 2 or SEQ ID NO: 3.

The targeting peptides can be subject to various changes, substitutions, insertions, and deletions where such changes provide for certain advantages in its use. In this regard, targeting peptides that bind to and/or complex with a proteolytically cleaved extracellular portion of an Ig superfamily cell adhesion molecule can be substantially homologous with, rather than be identical to, the sequence of a recited polypeptide where one or more changes are made and it retains the ability to function as specifically binding to and/or complexing with the proteolytically cleaved extracellular portion of an Ig superfamily cell adhesion molecule.

The targeting peptides can be in any of a variety of forms of polypeptide derivatives, that include amides, conjugates with proteins, cyclized polypeptides, polymerized polypeptides, analogs, fragments, chemically modified polypeptides, and the like derivatives.

The term “analog” includes any polypeptide having an amino acid residue sequence substantially identical to a sequence specifically shown herein in which one or more residues have been conservatively substituted with a functionally similar residue and that specifically binds to and/or complexes with the proteolytically cleaved extracellular portion of an Ig superfamily CAM as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein also include any polypeptide having one or more additions and/or deletions or residues relative to the sequence of a polypeptide whose sequence is shown herein, so long as the requisite activity is maintained.

The term “fragment” refers to any subject polypeptide having an amino acid residue sequence shorter than that of a polypeptide whose amino acid residue sequence is shown herein.

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

The targeting peptides can be synthesized by any of the techniques that are known to those skilled in the peptide art, including recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

It will be appreciated that the targeting peptide can bind to and/or complex with homophilic binding domains of proteolytically cleaved extracellular fragments of other Ig superfamily cell adhesion molecules, besides PTPs. For example, a similar molecular detection strategy described herein can be used with any other Ig superfamily CAM having a homophilic binding cell surface protein whose ligand binding site is known. A large variety of cell surface proteins, including other phosphatases, are cleaved at the cell surface (Streuli M, Saito H (1992) Expression of the receptor-linked protein tyrosine phosphatase LAR: proteolytic cleavage and shedding of the CAM-like extracellular region. EMBO J 11:897-907; Anders L, Ullrich A (2006) Furin-, ADAM 10-, and gamma-secretase-mediated cleavage of a receptor tyrosine phosphatase and regulation of beta-catenin's transcriptional activity. Mol Cell Biol 26:3917-3934; Haapasalo A, Kovacs D M (2007) Presenilin/gamma-secretase-mediated cleavage regulates association of leukocyte-common antigen-related (LAR) receptor tyrosine phosphatase with beta-catenin. J Biol Chem 282:9063-9072; Chow J P, Noda M (2008) Plasmin-mediated processing of protein tyrosine phosphatase receptor type Z in the mouse brain. Neurosci Lett 442:208-212; Craig S E, Brady-Kalnay S M. Tumor-derived extracellular fragments of receptor protein tyrosine phosphatases (RPTPs) as cancer molecular diagnostic tools. Anticancer Agents Med Chem. 2011 January; 11(1):133-40. Review. PubMed PMID: 21235433; PubMed Central PMCID: PMC3337336; Craig S E, Brady-Kalnay S M. Cancer cells cut homophilic cell adhesion molecules and run. Cancer Res. 2011 Jan. 15; 71(2):303-9. Epub 2010 Nov. 17. PubMed PMID: 21084269; PubMed Central PMCID: PMC3343737; Phillips-Mason P J, Craig S E, Brady-Kalnay S M. Should I stay or should I go? Shedding of RPTPs in cancer cells switches signals from stabilizing cell-cell adhesion to driving cell migration. Cell Adh Migr. 2011 Jul. 1; 5(4):298-305. Epub 2011 Jul. 1. PubMed PMID: 21785275; PubMed Central PMCID: PMC3210297). These proteins represent additional targets for that can be readily used by the skilled artisan for forming therapeutic polypeptides that can be used to treat cancers (Barr A J, Ugochukwu E, Lee W H, King O N, Filippakopoulos P, Alfano I, Savitsky P, Burgess-Brown N A, Muller S, Knapp S (2009) Large-scale structural analysis of the classical human protein tyrosine phosphatome. Cell 136:352-363).

In some embodiments, the targeting peptide of an agent described herein can be directly linked to at least one of a detectable moiety, therapeutic agent, or theranostic agent. In alternative embodiments, the targeting peptide can be linked to at least one of a detectable moiety, therapeutic agent, or theranostic agent via a linking molecule.

For example, additional residues may also be added at either terminus of a targeting peptide for the purpose of providing a “linker” by which the targeting peptides can be conveniently linked and/or affixed to other detectable moieties, therapeutic agents, theranostic agents, polypeptides, proteins, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In some embodiments, the linking molecule may be a single amino acid linker. In an exemplary embodiment, the amino acid residue linker is a lysine residue.

In addition, a subject polypeptide can differ by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linking molecule is selected in part based on its ability to alter the phobicity (e.g., to cause the molecular probe to become more hydrophilic or hydrophobic) depending on its desired use.

In some embodiments, the linker can be a flexible peptide linker that links the targeting peptide to other polypeptides, proteins, and/or molecules, such as detectable moieties, labels, therapeutic agents, theranostic agents, solid matrices, or carriers. A flexible peptide linker can be about 20 or fewer amino acids in length. For example, a peptide linker can contain about 12 or fewer amino acid residues, e.g., 1, 2, 3, 4, 5, 6, 7, 8, 9, 10, 11, and 12. In some cases, a peptide linker comprises two or more of the following amino acids: glycine, serine, lysine, alanine, and threonine. Where the linker is a peptide linker, the polypeptide-linker may be produced as a single recombinant polypeptide using a conventional molecular biological/recombinant DNA method.

In some embodiment, where the agent is used a molecular probe for detection of cancer or cancer cells, the targeting peptide can be linked to a chelating agent of a detectable moiety via a linking molecule which is not a contiguous portion of either the targeting peptide or chelating agent and which covalently joins an amino acid of the targeting peptide to a carboxyl group of the chelating agent, e.g., DOTA. As used herein, a linking molecule that is “not a contiguous portion” means that the targeting peptide and detectable moiety are connected via an additional element that is not a part of the targeting peptide or a portion of the detectable moiety that is contiguous in nature and functions as a linker.

Alternatively, a linking molecule may be a non-amino or non-peptide linker. As used herein, a non-peptide linker useful for the method described herein is a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. Typically such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

For the production of recombinant polypeptides, a variety of host organisms may be used. Examples of hosts include, but are not limited to: bacteria, such as E. coli, yeast cells, insect cells, plant cells and mammalian cells. Choice of a host organism will depend on the particular application of the polypeptide-targeted detectable moiety, therapeutic or theranostic agent. The skilled artisan will understand how to take into consideration certain criteria in selecting a suitable host for producing the recombinant polypeptide. Factors affecting selection of a host include, for example, post-translational modifications, such as phosphorylation and glycosylation patterns, as well as technical factors, such as the general expected yield and the ease of purification. Host-specific post-translational modifications of the polypeptide-targeted detectable moiety, therapeutic or theranostic agent, which is to be used in vivo, should be carefully considered because certain post-translational modifications are known to be highly immunogenic (antigenic).

In some embodiments, the targeting peptide can be directly or indirectly labeled with a detectable moiety. The detectable moiety can include any contrast agent or detectable label that facilitate the detection step of a diagnostic or therapeutic method by allowing visualization of the complex formed by binding of the molecular probe comprising the targeting peptide and detectable moiety and/or theranostic agent to the proteolytically cleaved extracellular fragment of the Ig superfamily cell adhesion molecule. The detectable moiety can be selected such that it generates a signal, which can be measured and whose intensity is related (preferably proportional) to the amount of the molecular probe bound to the tissue being analyzed. Methods for labeling biological molecules, such as polypeptides are well-known in the art.

Any of a wide variety of detectable moieties can be linked with the targeting peptides in a molecular probe described herein. Examples of detectable moieties include, but are not limited to: various ligands, radionuclides, fluorescent agents and dyes, infrared and near infrared agents, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, biotin, dioxigenin or other haptens and proteins for which antisera or monoclonal antibodies are available.

In some embodiments, the molecular probes described herein may be used in conjunction with non-invasive imaging (e.g., neuroimaging) techniques for in vivo imaging of the molecular probe, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a labeled molecular probe, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of molecular probe along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

In one example, the detectable moiety can include a radiolabel, that is directly or indirectly linked (e.g., attached or complexed) with the targeting peptide using general organic chemistry techniques. The detectable peptide can also include radiolabels, such as ⁶⁸Ga, ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S, for PET by techniques well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, N Y 1986) the contents of which are hereby incorporated by reference. The detectable moiety can also include ¹²³I for SPECT. The ¹²³I can be coupled to the targeting peptide can by any of several techniques known to the art. See, e.g., Kulkarni, Int. J. Rad. Appl. & Inst. (Part B) 18: 647 (1991), the contents of which are hereby incorporated by reference. In addition, detectable moiety can include any radioactive iodine isotope, such as, but not limited to ¹³¹I, ¹²⁵I, or ¹²³I. The radioactive iodine isotopes can be coupled to the targeting peptide by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art.

The detectable moiety can further include known metal radiolabels, such as Technetium-99m (^{99m}Tc), ¹⁵³Gd, ¹¹¹In, ⁷⁶Ga, ²⁰¹Tl, ⁸²Rb, ⁶⁴Cu, ⁹⁰Y, ¹⁸⁸Rh T(tritium), ¹⁵³Sm, ⁸⁹Sr, and ²¹¹At. Modification of the targeting peptid to introduce ligands that bind such metal ions can be effected without undue experimentation by one of ordinary skill in the radiolabeling art. The metal radiolabeled molecular probes can then be used to detect cancers, such as GBM in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

In the some embodiments, the detectable moiety can include a chelating agent (with or without a chelated radiolabel metal group). Exemplary chelating agents can include those disclosed in U.S. Pat. No. 7,351,401, which is herein incorporated by reference in its entirety. In some embodiments, the chelating agent is 1,4,7,10-tetraazacyclododecane-1,4,7,10-tetraacetic acid (DOTA).

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR800, and QUASAR 670. Fluorescent labeling agents can include other known fluorophores, or proteins known to the art, for example, green fluorescent protein. The disclosed targeting peptides can be directly or indirectly coupled to the fluorescent labeling agents, administered to a subject or a sample, and the subject/sample examined by fluorescence spectroscopy or imaging to detect the labeled compound.

In some embodiments, the detectable moiety includes a fluorescent dye. Exemplary Fluorescent dyes include fluorescein isothiocyanate, cyanines such as Cy5, Cy5.5 and analogs thereof (e.g., sulfo-Cyanine 5 NHS ester and Cy5.5 maleimide). See also Handbook of Fluorescent Probes and Research Chemicals, 6^{th} Ed., Molecular Probes, Inc., Eugene Oreg., which is incorporated herein by reference.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13: 195-208; and Cancer 67: 1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor particles, can be employed in a molecular probe as described in Gao, et al “In vivo cancer targeting and imaging with semiconductor quantum dots”, Nature Biotechnology, 22, (8), 2004, 969-976, the entire teachings of which are incorporated herein by reference. The disclosed targeting peptides can be coupled to the quantum dots, administered to a subject or a sample, and the subject/sample examined by fluorescence spectroscopy or imaging to detect the labeled compound.

In certain embodiments, a detectable moiety includes a MRI contrast agent. MRI relies upon changes in magnetic dipoles to perform detailed anatomic imaging and functional studies. MRI can employ dynamic quantitative T₁ mapping as an imaging method to measure the longitudinal relaxation time, the T₁ relaxation time, of protons in a magnetic field after excitation by a radiofrequency pulse. T₁ relaxation times can in turn be used to calculate the concentration of a molecular probe in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed targeting peptides can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium(III)chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, the molecular probe can include a targeting peptide that can be coupled or linked to a chelating agent and a single metal radiolabel. For example, the molecular probe can have the formula (I):

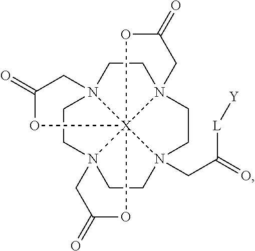

wherein X is Ga or Gd metal ion, L is a linking molecule, and Y is a polypeptide having an amino acid sequence selected from the group consisting of SEQ ID NO: 4, SEQ ID NO: 5, SEQ ID NO: 6, SEQ ID NO: 7, and SEQ ID NO: 8.

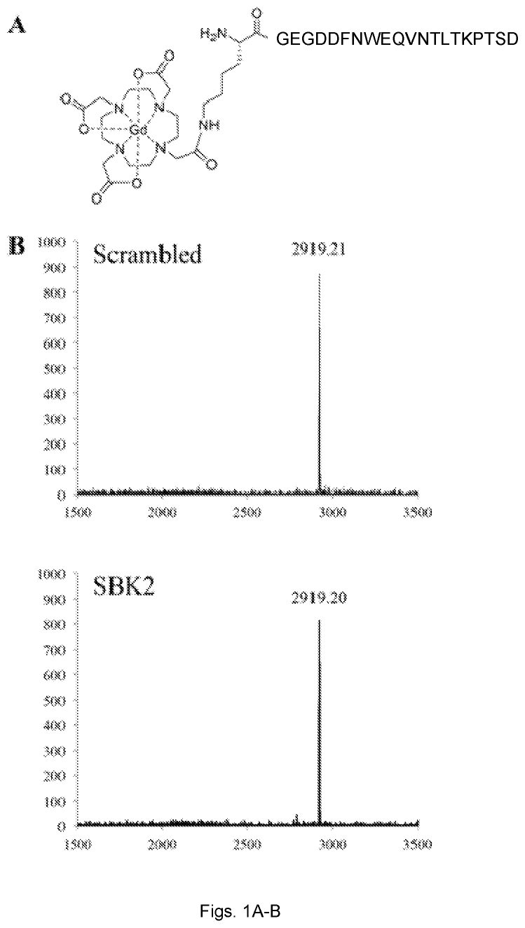

In certain embodiments, L can be a single amino acid residue, such as lysine and Y is a polypeptide having an amino acid sequence SEQ ID NO: 5. This molecular probe can be prepared by coupling a macrocyclic chelator DOTA attached to a lysine with the N-terminal glycine of the PTPμ targeted peptide having the amino acid SEQ ID NO:5 (SBK2) via an amide bond (see FIG. 1A).

The molecular probe described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the molecular probe can be by intravenous injection of the molecular probe in the subject. Single or multiple administrations of the probe can be given. “Administered”, as used herein, means provision or delivery of a molecular probe in an amount(s) and for a period of time(s) effective to label cancer cells in the subject.

Molecular probes described herein can be administered to a subject in a detectable quantity of a pharmaceutical composition containing a molecular probe or a pharmaceutically acceptable water-soluble salt thereof, to a patient.

Formulation of the molecular probe to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A “detectable quantity” means that the amount of the detectable compound that is administered is sufficient to enable detection of binding of the compound to the cancer cells. An “imaging effective quantity” means that the amount of the detectable compound that is administered is sufficient to enable imaging of binding of the molecular probe to the cancer cells.

The molecular probes administered to a subject can be used in a method to detect and/or determine the presence, location, and/or distribution of cancer cells, i.e., cancer cells associated with proteolytically cleaved extracellular fragments of Ig superfamily cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the molecular probe in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the molecular probe may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the composition. In this case, the baseline and post-administration images can be compared to ascertain the presence, absence, and/or extent of a particular disease or condition.

In one aspect, the molecular probes may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the molecular probes can be given intravenously about 24 hours prior to pre-surgical stereotactic localization MRI. The molecular probes can be imaged on gradient echo MRI sequences as a contrast agent that localizes with the glioma.

Molecular probes described herein that specifically bind to and/or complex with proteolytically cleaved Ig superfamily cell adhesion molecules (PTPμ) associated with cells can be used in intra-operative imaging (IOI) techniques to guide surgical resection and eliminate the “educated guess” of the location of the tumor margin by the surgeon. Previous studies have determined that more extensive surgical resection improves patient survival Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Thus, molecular probes that function as diagnostic molecular imaging agents have the potential to increase patient survival rates.

In some embodiments, to identify and facilitate removal of cancers cells, microscopic intra-operative imaging (IOI) techniques can be combined with systemically administered or locally administered molecular probes described herein. The molecular probe upon administration to the subject can target and detect and/or determine the presence, location, and/or distribution of cancer cells, i.e., cancer cells associated with proteolytically cleaved extracellular fragments of Ig superfamily cell adhesion molecules, in an organ or body area of a patient. In one example, the molecular probe can be combined with IOI to identify malignant cells that have infiltrated and/or are beginning to infiltrate at a tumor brain margin. The method can be performed in real-time during brain or other surgery. The method can include local or systemic application of the targeted molecular probe described herein that includes a detectable moiety, e.g., a fluorescent or MRI contrast moiety. An imaging modality can then be used to detect and subsequently gather image data. The imaging modality can include one or combination of known imaging techniques capable of visualizing the molecular probe. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micromachine).

In one example, a targeted molecular probe linked to a fluorescent detectable moiety can be topically applied as needed during surgery to interactively guide a surgeon and/or surgical instrument to remaining abnormal cells. The probe may be applied locally in low concentration, making it unlikely that pharmacologically relevant concentrations are reached. In one example, excess material may be removed (e.g., washed off) after a period of time (e.g., incubation period).

Another embodiment described herein relates to a method of monitoring the efficacy of a cancer therapeutic or cancer therapy administered to a subject. The methods and agents described herein can be used to monitor and/or compare the invasion, migration, dispersal, and metastases of a cancer in a subject prior to administration of a cancer therapeutic or cancer therapy, during administration, or post therapeutic regimen.

A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The cancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA.

The method of monitoring the efficacy of a cancer therapeutic can include the steps of administering in vivo to the animal a molecular probe as described herein, then visualizing a distribution of the molecular probe in the animal (e.g., with an in vivo imaging modality as described herein), and then correlating the distribution of the molecular probe with the efficacy of the cancer therapeutic. It is contemplated that the administering step can occur before, during, and after the course of a therapeutic regimen in order to determine the efficacy of a chosen therapeutic regimen. One way to assess the efficacy of the cancer therapeutic is to compare the distribution of a molecular probe pre and post cancer therapy.

In some embodiments, the molecular probe bound to and/or complexed with the proteolytically cleaved extracellular fragment of the Ig superfamily cell adhesion molecule is detected in the subject to detect and/or provide the location and/or distribution of the cancer cells in the subject. The location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the molecular probe to the subject and detecting the molecular probe bound to and/or complexed with cancer cells in the subject prior to administration of the cancer therapeutic and/or cancer therapy.

In certain embodiments, the methods and molecular probes described herein can be used to measure the efficacy of a therapeutic administered to a subject for treating a metastatic, invasive, or dispersed cancer. In this embodiment, the molecular probe can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the molecular probe can be used to define the distribution of the metastatic cancer pre-operative and post-operative to determine the efficacy of the surgical resection. Optionally, the methods and molecular probes can be used in an intra-operative surgical procedure as describe above, such as a surgical tumor resection, to more readily define and/or image the cancer cell mass or volume during the surgery.

In other embodiments, an agent for treating cancer can include a theranostic agent or therapeutic agent, which can be directly or indirectly linked to the targeting peptide. In one example, the theranostic or therapeutic agent linked to the targeting peptide cancer can be used in a photodynamic therapy to treat cancer or tumors (e.g., brain cancer or tumors). Photodynamic therapy (PDT) is a site specific treatment modality that requires the presence of a photosensitizer, light, and adequate amounts of molecular oxygen to destroy targeted tumors (Grossweiner, Li, The science of phototherapy. Springer: The Netherlands, 2005). Upon illumination, a photoactivated sensitizer transfers energy to molecular oxygen that leads to the generation of singlet oxygen (O²) and other reactive oxygen species (ROS), which initiate apoptosis and oxidative damage to cancer cells. Only the cells that are exposed simultaneously to the theranostic PDT drug (which is non-toxic in the dark) and light are destroyed while surrounding healthy, non-targeted and nonirradiated cells are spared from photodamage. Furthermore, the fluorescence of the photosensitizer molecules enables simultaneous diagnostic optical imaging that can be used to guide the PDT cancer treatment.

Methods for conducting photodynamic therapy are known in the art. See for example Thierry Patrice. Photodynamic Therapy; Royal Society of Chemistry, 2004. A pharmaceutical composition including an agent, which comprises a targeting peptide and a theranostic agent directly or indirectly linked to a targeting peptide, can be applied to an organ or tissue as a step in PDT. In certain embodiments, the composition is applied to an epithelial, mesothelial, synovial, fascial, or serosal surface, including, but not limited to, the eye, esophagus, mucous membrane, bladder, joint, tendon, ligament, bursa, gastrointestinal, genitourinary, pleural, pericardial, pulmonary, or uroepithelial surfaces.

A theranostic agent or therapeutic agent for PDT directly or indirectly linked to the targeting peptide can be administered to a subject with cancer by systemic administration, such as intravenous administration. Upon administration, the targeted agent can localize to and/or accumulate at the site of the targeted tumor or cancer. In some embodiments, specific binding and/or complexing with a proteolytically cleaved extracellular fragment of an immunoglobulin (Ig) superfamily cell adhesion molecule that is expressed by a cancer cell or another cell in the cancer cell microenvironment allows the agent including the targeting peptide and the PDT agent to be bound to, complexed with and/or taken up by the targeted cells by, for example, endocytosis. This binding and/or uptake is specific to the targeted cells, which allows selective targeting of the cancer cells and/or cells in the cancer cell microenvironment in the subject by the targeted agents.

Following administration and localization of the targeting peptide linked to a theranostic agent or therapeutic agent to the targeted cancer cells, the targeted cancer cells can be exposed to therapeutic amount of light that causes cancer cell damage and/or suppression of cancer cell growth. The light, which is capable of activating the PDT agent can delivered to the targeted cancer cells using, using for example, semiconductor laser, dye laser, optical parametric oscillator or the like. It will be appreciated that any source light can be used as long as the light excites the hydrophobic PDT agent.

By way of example, agents including a targeting peptide and a PDT agent can provide image guidance for glioma tumor resection and allow for subsequent PDT to eliminate unresectable or remaining cancer cells. In certain embodiments, the targeting moiety can comprise a peptide having SEQ ID NO:5.

PDT agent photosensitizer compounds for use in an agent described herein can include compounds that are excited by an appropriate light source to produce radicals and/or reactive oxygen species. Typically, when a sufficient amount of photosensitizer appears in diseased tissue (e.g., tumor tissue), the photosensitizer can be activated by exposure to light for a specified period. The light dose supplies sufficient energy to stimulate the photosensitizer, but not enough to damage neighboring healthy tissue. The radicals or reactive oxygen produced following photosensitizer excitation kill the target cells (e.g., cancer cells). In some embodiments, the targeted tissue can be locally illuminated. For example, light can be delivered to a photosensitizer via an argon or copper pumped dye laser coupled to an optical fiber, a double laser consisting of KTP (potassium titanyl phosphate)/YAG (yttrium aluminum garnet) medium, LED (light emitting diode), or a solid state laser.

PDT sensitizers for use as a theranostic or therapeutic agent can include a first generation photosensitizer (e.g., hematoporphyrin derivatives (HpDs) such as Photofrin (porfimer sodium), Photogem, Photosan-3 and the like). In some embodiments, PDT sensitizers can include second and third generation photosensitizers such as porphyrinoid derivatives and precursors. Porphyrinoid derivatives and precursors can include porphyrins and mettaloporphrins (e.g., meta-tetra(hydroxyphenyl)porphyrin (m-THPP), 5,10,15,20-tetrakis(4-sulfanatophenyl)-21H,23H-porphyrin (TPPS₄), and precursors to endogenous protoporphyrin IX (PpIX): 5-aminolevulinic acid (5-ALA, which has been used for photodynamic therapy (PDT) of gliomas with some success (Stummer, W. et al. J Neurooncol. 2008, 87(1):103-9.).), methyl aminolevulinate (MAL), hexaminolevulinate (HAL)), chlorins (e.g., benzoporphyrin derivative monoacid ring A (BPD-MA), meta-tetra(hydroxyphenyl)chlorin (m-THPC), N-aspartyl chlorin e6 (NPe6), and tin ethyl etiopurpurin (SnET2)), pheophorbides (e.g., 2-(1-hexyloxyethyl)-2-devinyl pyropheophorbide (HPPH)), bacteriopheophorbides (e.g., bacteriochlorphyll a, WST09 and WST11), Texaphyrins (e.g., motexafin lutetium (Lu-Tex)), and phthalocyanines (PCs) (e.g., aluminum phthalocyanine tetrasulfonate (AlPcS4) and silicon phthalocyanine (Pc4)). In some embodiments, the PDT sensitizer can include cationic zinc ethynylphenyl porphyrin. Although porphyrinoid structures comprise a majority of photosensitizers, several non-porphyrin chromogens exhibit photodynamic activity. These compounds include anthraquinones, phenothiazines, xanthenes, cyanines, and curcuminoids.

In some embodiments, a theranostic agent or therapeutic agent described herein can include a phthalocyanine compound. Phthalocyanines, hereinafter also abbreviated as “Pcs”, are a group of photosensitizer compounds having the phthalocyanine ring system. Phthalocyanines are azaporphyrins consisting of four benzoindole groups connected by nitrogen bridges in a 16-membered ring of alternating carbon and nitrogen atoms (i.e., C₃₂H₁₆N₈) which form stable chelates with metal and metalloid cations. In these compounds, the ring center is occupied by a metal ion (either a diamagnetic or a paramagnetic ion) that may, depending on the ion, carry one or two ligands. In addition, the ring periphery may be either unsubstituted or substituted. Phthalocyanines strongly absorb clinically useful red or near IR radiation with absorption peaks falling between about 600 and 810 nm, which potentially allows deep tissue penetration by the light. The synthesis and use of a wide variety of phthalocyanines in photodynamic therapy is described in International Publication WO 2005/099689.

In some embodiments, the phthalocyanine compound is Pc4. Pc4 is relatively photostable and virtually non-toxic. In some embodiments, the phthalocyanine compound is an analog of the PDT photosensitizing drug Pc4 found to be effective in targeted bioimaging and targeted PDT of cancer in a subject, see for example, U.S. Pat. No. 9,889,199, the contents of which are hereby incorporated by reference. In some embodiments, the Pc4 analog can include Pc413.

In some embodiments, a theranostic agent or therapeutic agent, such as a phthalocyanine compound can be linked to the targeting peptide via a linker. The linker can be of any suitable length and contain any suitable number of atoms and/or subunits. The linker can include one or combination of chemical and/or biological moieties. Examples of chemical moieties can include alkyl groups, methylene carbon chains, ether, polyether, alkyl amide linkers, alkenyl chains, alkynyl chains, disulfide groups, and polymers, such as poly(ethylene glycol) (PEG), functionalized PEG, PEG-chelant polymers, dendritic polymers, and combinations thereof. Examples of biological moieties can include amino acid residues, peptides, modified peptides, streptavidin-biotin or avidin-biotin, polyaminoacids (e.g., polylysine), polysaccharides, glycosaminoglycans, oligonucleotides, phospholipid derivatives, and combinations thereof.

In other embodiments, agents for treating cancer can include a therapeutic agent linked to a targeting peptide. The targeting peptide can be coupled or linked to the therapeutic agent using a linking molecule. The linking molecule may be an amino acid residue or peptide linker. Alternatively, a linking molecule may be a non-peptide linker. The therapeutic agent can include an anti-cancer or an anti-proliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be directly or indirectly linked to a targeting peptide in a molecular probe described herein include Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer therapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosamOinoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other anti-cancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Still other anti-cancer therapeutic agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In some embodiments, cytotoxic compounds are included in a molecular probe described herein. Cytotoxic compounds include small-molecule drugs such as doxorubicin, mitoxantrone, methotrexate, and pyrimidine and purine analogs, referred to herein as antitumor agents.

The agents including a targeting peptide linked to a therapeutic agent described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The agents including a targeting peptide linked to a therapeutic agent described herein can be administered alone as a monotherapy, or in conjunction with or in combination with one or more additional therapeutic agents. For example, the agent including a targeting peptide linked to a therapeutic agent described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The agent including a targeting peptide linked to a therapeutic agent described herein and additional therapeutic agent can be components of separate pharmaceutical compositions, which can be mixed together prior to administration or administered separately. The agent including a targeting peptide linked to a therapeutic agent described herein can, for example, be administered in a composition containing the additional therapeutic agent, and thereby, administered contemporaneously with the agent. Alternatively, the agent including a targeting peptide linked to a therapeutic agent described herein can be administered contemporaneously, without mixing (e.g., by delivery of the agent on the intravenous line by which the therapeutic agent is also administered, or vice versa). In another embodiment, the agent including a targeting peptide linked to a therapeutic agent described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The agent including a targeting peptide linked to a therapeutic agent described herein (or composition containing the agent) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the molecular probe and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the detectable moiety, therapeutic or theranostic agent in the subject, the agent can be administered between, for example, once a day or once a week.

For example, the administration of the disclosed molecular probe and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The disclosed agent including a targeting peptide linked to a therapeutic agent described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of disclosed agent including a targeting peptide linked to a therapeutic agent described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the agent including a targeting peptide linked to a therapeutic agent described herein can also depend on the disease state or condition being treated along with the clinical factors and the route of administration of the compound.

The disclosed agent and/or additional therapeutic agent described herein can be administered to the subject in conjunction with an acceptable pharmaceutical carrier or diluent as part of a pharmaceutical composition for therapy. Formulation of the compound to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The following examples are included to demonstrate preferred embodiments.

### Example 1

In this example, we describe the generation of a PTPμ-targeted, peptide-based molecular imaging contrast agent with a single Gd, SBK2-Lys-(Gd-DOTA) that was prepared using conventional chemical synthesis methods and which can be readily used with other targeting peptides. Very few examples of molecular agents exist in the literature that utilize a single Gd ion and effectively enhance a tumor target. Most of these studies use either a change in MR signal derived from qualitative images or long acquisition times that do not allow for dynamic quantitative comparisons. Even fewer examples exist where these single Gd molecular agents have been subjected to quantitative MR techniques that allow for comparisons of sensitivity. For the studies described here, T₁ mapping was utilized in order to allow absolute T₁ relaxation values to be determined. T₁ mapping has recently garnered intense interest in Cardiovascular Magnetic Resonance (CMR) due to its ability to provide direct T₁ values, which lead to improved tissue characterization by MRI. In addition, we wanted to utilize a dynamic acquisition method to allow us to determine whether these agents differed in their ability to bind to a particular location or whether they simply flowed through the tissue. Specific binding events between a ligand/receptor occur within minutes and change over time. We demonstrate here the use of a molecular imaging agent with a dynamic quantitative T₁ mapping MR method that allows for calculation of Gd concentration over time. We show in both heterotopic and orthotopic mouse models of glioma that the single Gd molecular contrast agent, SBK2-Lys-(Gd-DOTA), displays sustained binding to tumors in comparison to a non-specific peptide-based control agent. For the first time, our dynamic quantitative MRI data show that use of a single Gd, when combined with an appropriate peptide, generates a molecular contrast agent that specifically recognizes tumors in vivo. The single Gd molecular contrast agent exhibits sustained binding and Gd retention in tumors thus enhancing detection compared to a non-specific agent using MRI.

### Experimental Section

### Reagents

The Fmoc-protected amino acids, 2-chlorotrityl chloride resin, (0-(6-chlorobenzotriazol-1-yl)-N,N,N′,N′-tetramethyluronium hexafluorophosphate (HCTU) and benzotriazol-1-yl-oxy-tris-(pyrrolidino) phosphonium hexafluorophosphate (PyBOP) used for peptide synthesis were purchased from Chem-Impex International, Inc. (Wood Dale, Ill., USA). The source of the following chemicals are: Sigma-Aldrich (St. Louis, Mo., USA) for anhydrous N,N-diisopropylethyl amine (DIPEA), trifluoroacetic acid (TFA), 2,2′-(ethylenedioxy)diethanethiol (DODT), triisopropylsilane, 4-methyl piperidine, α-cyano-4-hydroxycinnamic acid (CHCA), ammonium bicarbonate, ammonium acetate and meglumine; Fisher Scientific (Pittsburgh, Pa., USA) for N,N-dimethylformamide (DMF) and dichloromethane; Apex Bio Technology (Houston, Tex., USA) for anhydrous 1-hydroxybenzotriazole (HOBt); Strem Chemicals (Newburyport, Mass., USA) for gadolinium (III) acetate tetrahydrate; Macrocyclics, Inc. (Plano, Tex., USA) for Fmoc-L-Lys-mono-amide-DOTA-tris (t-Bu ester). Multihance® was purchased from Bracco Diagnostics, Optimark® was purchased from Mallinckrodt Pharmaceuticals (St. Louis, Mo., USA), Magnevist® was purchased from Bayer Healthcare Pharmaceuticals, Inc. (Wayne, N.J., USA), and saline was obtained from Hospira, Inc. (Lake Forest, Ill., USA).

### Synthesis and Characterization of SBK2-Lys-(Gd-DOTA) and Scrambled-Lys-(Gd-DOTA) Agents

Synthesis of the PTPμ targeted peptide, SBK2 (CGEGD-DFNWEQVNTLTKPTSD) SEQ ID NO: 8), and a Scrambled control peptide (GTQDETGNFDWPVSEDLNKT) SEQ ID NO: 9) was done using conventional solid-phase synthesis methods and FMOC-protected amino acids on a CS Bio CS336X automated peptide synthesizer using HCTU as the coupling agent. Fmoc-L-Lys-mono-amide-DOTA-tris (t-Bu ester) was manually coupled to each peptide using PyBOP and HOBt as the coupling agents. Purity was assessed using analytical RP-HPLC with an Eclipse XDB-C18, 4.6×150 mm, 5 μm column (Agilent Technologies, Santa Clara, Calif., USA). By HPLC, purity of the peptides was >99%. Matrix-assisted laser desorption/ionization time-of-flight (MALDI-TOF; Autoflex Speed, Bruker Corp., Billerica, Mass., USA) using CHCA as a matrix was used to confirm molecular weights. Complexation reactions were carried out at 37° C. over several days with constant monitoring to maintain the pH of the reactions between 5.0 and 6.0 in order to balance the solubility of the acidic SBK2 and Scrambled peptides with the solubility of Gd acetate. For these reactions, the peptide conjugates were dissolved in 0.05 M ammonium bicarbonate to obtain 20 mg/mL, pH 5.5-6.0 solutions. 1-2 molar equivalents of Gd acetate tetrahydrate, 100 mM, pH 5.0 were gradually added over time until 10 molar equivalents of Gd in total were added. Completeness of the complexation reactions was assessed by MALDI-TOF using CHCA as a matrix. Once complete, the pH of the reactions was raised to 8.0 with ammonium bicarbonate to precipitate any uncomplexed Gd and centrifuged (10 min, room temperature, 2800×g). The supernatants were loaded into cellulose ester dialysis membranes, MWCO 500-1000D (Spectrum Laboratories, Inc., Rancho Dominguez, Calif., USA) and dialyzed extensively. Two exchanges of 50 mM ammonium acetate buffer were followed by one exchange with 50 mM ammonium bicarbonate buffer, one exchange with 20 mM ammonium bicarbonate and two exchanges with 10 mM ammonium bicarbonate. The dialyzed products were filtered through 0.2 μm filters and lyophilized. The Gd content of the final products was measured using inductively coupled plasma optical emission spectroscopy (ICP-OES) (Agilent 730 Axial ICP-OES; Agilent Technologies, Wilmington, Del., USA). In addition, Arsenazo III was used to confirm the absence of free Gd. The molecular weights of the final products were confirmed by MALDI-TOF as shown in FIG. 1. The predicted molecular weight of both the SBK2 and Scrambled agent is 2922.13. By MALDI-TOF, (m/z, M⁺) for the SBK2 agent was observed to be 2919.20, and for the Scrambled agent, 2919.21.

T₁ relaxation constants for the agents were measured on the Bruker Biospec 9.4 T MRI scanner (Bruker Corp., Billerica, Mass., USA) at 37° C. using the T₁ mapping acquisition described previously. For this in vitro study, 100 signal averages were acquired for determination of the agent relaxivities (r₁). The T₁ relaxivities were obtained by measuring a dilution series of two lots of each agent on three separate occasions using saline as the diluent. A 60 MHz Bruker Minispec Relaxometer at 37° C. also was used to measure T₁ and T₂ relaxation constants. T₁ relaxation constants were measured with an inversion recovery pulse sequence and a Carr-Purcell-Meiboom-Gill sequence with 5000 data points was used to measure T₂ relaxation constants.

### Cell Culture and Flank Tumor Implants

The NIH athymic nude female mice were bred in the Athymic Animal Core Facility. The Institutional Animal Care and Use Committee approved all of the animal protocols. The human LN-229 glioma cell line from the American Type Culture Collection (Manassas, Va., USA) was stably-infected with lentivirus encoding green fluorescent protein (GFP) and cultured as previously described. The cells were diluted with BD Matrigel Matrix (BD Biosciences, Franklin Lakes, N.J., USA), prior to injecting 2×10⁶ cells into each right flank of the nude athymic mice (NCr-nu/+, NCr-nu/nu, 20-25 g each) as previously described.

### Cell Culture and Orthotopic Xenograft Intracranial Tumors

The CNS-1 cells, a gift from Mariano S. Viapiano, were cultured in RPMI medium supplemented with 5% fetal bovine serum. CNS-1 cells were infected with lentivirus to express GFP 48 h prior to harvesting. Cells were harvested for intracranial implantation by trypsinization and concentrated to 2.5×10⁴ cells/microliter of phosphate-buffered saline (PBS). Mice 6 to 7 weeks of age were anesthetized and prepared for intracranial injection as previously described. Cells were deposited at a rate of 1 μL per min for a total of 50,000 cells into the right striatum at a depth of 1.5-2 mm from the dura using a 10 μL syringe (26-gauge needle; Hamilton Co, Reno, Nev.). The needle was slowly withdrawn, bone wax (Fine Science Tools, Foster City, C, USA) was used to seal the hole, and the incision was closed with sutures. Mice were imaged as described below 7 days post tumor implantation.

Molecular Imaging of Tumors with MRI

We performed MRI using a Bruker Biospec 9.4 T preclinical MRI scanner (Bruker Corp., Billerica, Mass., USA) with a 35 mm inner diameter mouse body radiofrequency (RF) coil as described previously. Mice bearing LN-229 flank tumors were imaged at 4-8 weeks post-implantation for the heterotopic xenograft study. Mice implanted with orthotopic CNS-1 tumors were imaged 7 days post-implantation. Agents were dissolved in saline/20 U heparin, and 100 mM sterile meglumine was added as needed to modify the pH to between 7 and 8 and delivered intravenously as previously described. Mice were administered an equal concentration of Gd based on weight. Different doses from 0.1 to 0.2 mmol·Gd/kg, as indicated, were administered to flank tumor bearing mice. A group of 4 mice was used for each agent at each dose, except for Scrambled-Lys(Gd-DOTA) at 0.1 mmol/kg where 5 mice were used and SBK2-Lys-(Gd-DOTA) at 0.125 mmol/kg where 8 mice were used. For mice with intracranial CNS-1 tumors, 0.2 mmol·Gd/kg was used, and groups of 4 mice were used for each agent. After five baseline, pre-contrast, T₁ map scans, the targeted SBK2-Lys-(Gd-DOTA) agent or the non-targeted Scrambled-Lys-(Gd-DOTA) control was injected at the indicated dose in 150 μL followed by a 50 μL flush of saline. T₁ maps were consecutively acquired every 2.5 min over 62.5 min (flank tumors) or 45 min (intracranial tumors).

### Calculation of T₁ Mapping Values, Gd Concentration, and Clearance Rates

The analysis of T₁ relaxation times and calculation of Gd concentration has been described previously. Flank tumors were clearly visible in both the T₂-weighted and T₁ scans. Flank tumor regions of interest (ROIs) were manually outlined in MATLAB along with a “control” area where no obvious blood vessels or anatomic features were visible by either scan type. The same ROI for tumor and controls were then applied to all of the T₁ mapping images and the average T₁ map value was calculated. Normalized T₁ maps were calculated by dividing all T₁ relaxation times by the mean pre-contrast T₁ values. To calculate Gd concentrations, T₁ relaxivity constants determined at 9.4 T were used along with T₁ map values and equation 1:

$$C = \frac{( \frac{1}{{T}_{1} } + \frac{1}{{T}_{1 , pre}} )}{r 1}$$

where C is the concentration of Gd (mM), T₁ is the relaxation time (in seconds), T_{1,pre} is an average baseline T₁ map value (in seconds) and r₁ is the magnetic relaxivity constant for the contrast agent (mM⁻¹s⁻¹). All relaxivity constants are presented on a per Gd basis.

The clearance rates for the agents in flanks at each dose were determined by calculating the change in mean T₁ relaxation time over the time interval indicated after the agents were administered. A two-tailed Student's t-test was used to assess statistical significance.

As in our previous work, we calculated quantitative T₁ maps for the entire brain on a pixel-by-pixel basis and converted these values into heat maps to better visualize changes in T₁ relaxation times. To identify ROIs for tumor and contralateral control areas, the set of post-injection scans were averaged together and used to accurately draw both tumor and control ROIs that were then applied to each individual T₁ map scan. As with the flank tumors, the T₁ relaxation time maps were normalized and the clearance rates for each agent were calculated by determining the change in T₁ map value over time from 20 to 35 min after administering the agent.

### Results

Standard chemical synthesis methods were used to generate the PTPμ specific agent, SBK2-Lys-(Gd-DOTA), along with a non-specific control agent, Scrambled-Lys-(Gd-DOTA). Synthesis of these agents consisted of conventional solid phase peptide synthesis with commercially available Fmoc-L-Lys-mono-amide-DOTA-tris (t-Bu ester) added at the N-terminus of both SBK2 and Scrambled peptides. Following synthesis of the peptide-based conjugates, complexation with Gd acetate was performed, followed by extensive dialysis and lyophilization to generate the final products. FIG. 1 shows the structure of SBK2-Lys-(Gd-DOTA) which has a predicted molecular weight of 2922.13. MALDI-TOF spectra for both the specific and control agents are shown in panel B of FIG. 1. Gd content was measured using ICP-OES and lot specific measurements were used for dosing. At 9.4 T, the T₁ relaxivity for the SBK2-Lys-(Gd-DOTA) agent was 6.0±0.1 mM⁻¹s⁻¹ (mean±SE), and for Scrambled-Lys-(Gd-DOTA) agent, 5.9±0.1 mM⁻¹s⁻¹. For comparison, the clinical MRI contrast agents Optimark, Multihance, and Magnevist had the following T₁ relaxivity constants measured at 9.4 T: 4.5±0.1, 4.9±0.1, and 4.0±0.1 mM⁻¹s⁻¹, respectively. This simple synthesis contrasts with the more complex synthesis used to make the “first generation” PTPμ-based MR agent, SBK2-Tris-(Gd-DOTA)₃ which contained three Gd ions. For the previously published agents, a complex tris-propargyl linker was used and coupled to SBK2 or the control peptide. Following this step, three (Gd-DOTA) moieties were added using the copper-catalyzed azide-alkyne cycloaddition reaction.

We also measured the r₁ and r₂ constants at 60 MHz to compare the relaxivities of these single-Gd agents with the relaxivities we previously obtained for the three Gd agents on a per Gd basis. Both r₁ and r₂ for these single Gd agents were very similar to those measured for the larger three Gd agents with MW of 4664.62. At 60 MHz, the r₁ and r₂ constants (in mM⁻¹s⁻¹) were 8.4±0.1 and 10.1±0.2 for the SBK2-Lys-(Gd-DOTA) agent, and 8.5±0.1 and 9.9±0.3 for the Scrambled-Lys-(Gd-DOTA) agent. The r₁ and r₂ constants for SBK2-Tris-(Gd-DOTA)₃ were previously found to be 8.3 mM⁻¹s⁻¹ and 10.0 mM⁻¹s⁻¹, and for Scrambled-Tris-(Gd-DOTA)₃, the r₁ and r₂ constants were 8.7 mM⁻¹s⁻¹ and 10.8 mM⁻¹s⁻¹ respectively. Other investigators have reported obtaining similar r₁ constants for peptide-based molecular agents with one and three Gd ions.

Quantitative T₁ mapping was used to compare the ability of these single Gd agents to enhance heterotopic glioma xenograft LN-229 flank tumors. Our initial T₁ mapping experiments used a concentration of 0.2 mmol Gd/kg based upon our previous studies. We evaluated clearance rates over time between the SBK2 agent and that of the Scrambled agent. Distinct differences in clearance rate were observed between the two agents. SBK2 had a negative slope during a 10 minute interval occurring 10 to 20 minutes after injection which indicates binding of the SBK2 agent to its ligand during this time. At later time intervals, the clearance rates are positive as the agent clears out of the tumor. In contrast, the clearance rates for the Scrambled control agent are relatively constant and positive at all time intervals.

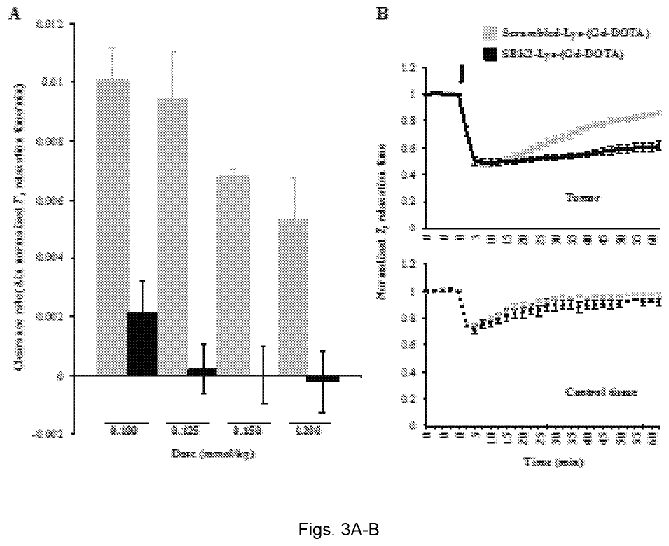

Next, four different doses of each agent, from 0.1 to 0.2 mmol Gd/kg, were evaluated to compare the specificity of the SBK2 agent to that of the Scrambled agent. As shown in FIG. 3A, at all 4 doses the clearance rate of the SBK2 agent between 15 and 30 min after injection was significantly slower than that of the Scrambled agent. These lower clearance rates demonstrate specific in vivo binding and retention of SBK2-Lys-(Gd-DOTA) in contrast to the non-specific Scrambled-Lys-(Gd-DOTA). In the clinical setting, prolonged enhancement by a specific agent would broaden the available imaging window and thus be of considerable practical use.

A typical clinical dose of a conventional agent such as Multihance is 0.1 mmol Gd/kg. FIG. 3B shows normalized T₁ relaxation times for both SBK2- and Scrambled-Lys-(Gd-DOTA) at 0.1 mmol/kg. Following 5 baseline scans, the agents were injected and a rapid drop in normalized T₁ is observed for both agents. This change in T₁ occurs at a similar rate and to a similar extent at all doses for the two agents (data not shown for the three higher doses). In addition to requiring a more difficult synthesis, the earlier SBK2 agent required twice the dose needed for SBK2-Lys-(Gd-DOTA), 0.2 mmol Gd/kg compared to 0.1 mmol Gd/kg to show specific binding and retention in flank tumors.

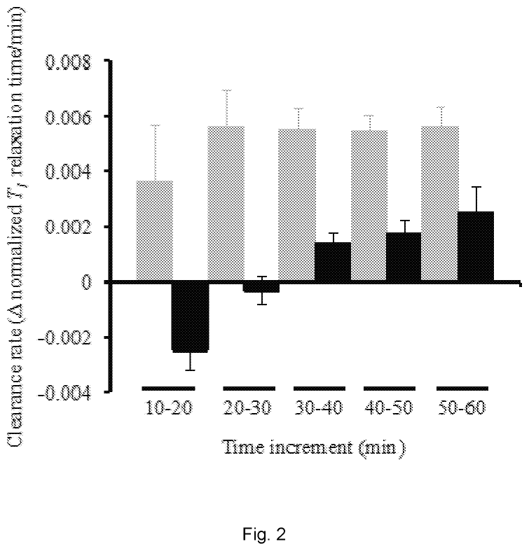

After an initial drop in T₁, both Gd-containing agents gradually clear out of non-tumor (“control”) tissue and return to baseline values as shown in the lower panel of FIG. 2B. Normalized T₁ relaxation times in tumor regions remain decreased for much longer in animals administered SBK2-Lys-(Gd-DOTA); however, compared to Scrambled-Lys-(Gd-DOTA). This suggests that SBK2-Lys-(Gd-DOTA), but not Scrambled-Lys-(Gd-DOTA), has specifically bound to its tumor-associated ligand and is retained over time. Based on experiments where T₁ values were monitored for up to two hours, we estimate that the Scrambled-Lys-(Gd-DOTA) agent would clear from the tumor region in about 2 hours while about 4 hours would be required for clearance of the SBK2-Lys-(Gd-DOTA) agent (data not shown).

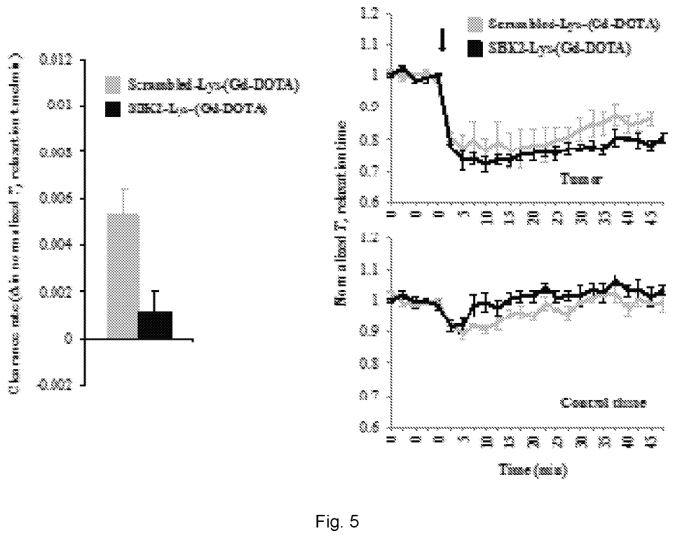

Finally, we tested the ability of the SBK2-Lys-(Gd-DOTA) to detect intracranial CNS-1 tumors. This tumor cell type is highly invasive and dispersive. Intracranial tumor centers were clearly visible by both T₂-weighted and T₁ scans; however, tumor margins were not possible to clearly distinguish. In contrast to imaging flank tumors, imaging of intracranial tumors is more difficult. By T₂-weighted, high-resolution MR images the average cross sectional area of CNS-1 brain tumors is 2.2 mm² (with a standard error of 0.3 mm²) as compared to 20.7 mm² (with a standard error of 2.8 mm²) for LN-229 flank tumors. Therefore, there is an almost 10 fold difference in size. It is likely that tumor recognition molecules, such as PTPμ, are present at a lower total number than in flank tumors simply due to size. In addition, circulating CAs have less access to an intracranial tumor due to the blood brain barrier and higher intratumoral pressure which restrict access. Nonetheless, we were able to specifically detect CNS-1 intracranial tumors with SBK2-Lys-(Gd-DOTA) (FIG. 5). As shown in FIG. 5, the clearance rates as calculated by the change in normalized T₁ relaxation value/time from 20 min to 35 min post injection are significantly different in the CNS-1 tumor-bearing animals receiving SBK2-Lys-Gd-DOTA compared to Scrambled-Lys-Gd-DOTA.

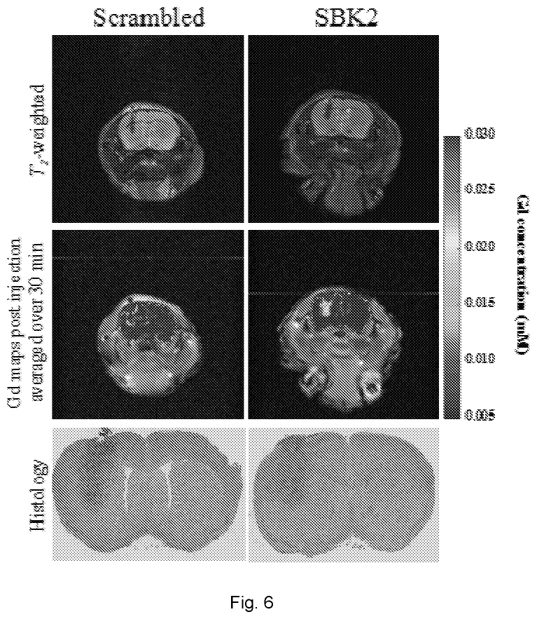

We were able to specifically detect CNS-1 intracranial tumors with SBK2-Lys-(Gd-DOTA), but this specific binding and corresponding slower clearance required 0.2 mmol Gd/kg. Gd concentration maps (mM) are shown in FIG. 5 along with High Resolution T₂-weighted images delineating anatomic detail. Gd concentrations are calculated based on absolute T₁ relaxation times pre- and post-contrast and in vitro r₁ relaxivity constants measured at 9.4 T as described in equation 1. Note that the non-specific Scrambled agent has crossed the compromised blood brain barrier, as has been documented for other Gd-based contrast agents (Wordworth et al), and accumulates in the main tumor mass. Despite administering twice the dose, the highest Gd concentration (0.03 mM) is about half that detected in flank tumors (max of 0.06 mM). FIG. 6 shows enhanced binding of the SBK2-Lys-(Gd-DOTA) agent to the tumor relative to the Scrambled-Lys-(Gd-DOTA) agent. Compared to the histology, the SBK2-Lys-(Gd-DOTA) highlights to a fuller extent this invasive tumor (FIG. 6).

To date, most of the single Gd-containing CAs belong to a family of molecules based on domain 1 of the cell adhesion molecule CD2, termed ProCA1-CD2. These CAs utilize optimized metal-binding characteristics of the CD2 domain 1 to promote specific binding to a Gd ion. This protein-based chelate can be coupled to peptides or affibodies to confer targeting capabilities. Biochemical features of the protein chelate, as well as its size, presumably contribute to the high relaxivity values measured. While in vitro and in vivo characterization of these protein-based chelates suggests many promising features, they have yet to be used with dynamic quantitative MR imaging methods in vivo. Without dynamic quantitative MR analyses, it is not yet possible to determine whether their characteristics translate into improved in vivo sensitivity and/or targeting compared to conventional agents. In terms of imaging glioma cells as opposed to angiography, there are limited examples of molecular CAs that cross the blood brain barrier with a single Gd chelate. One promising example involves the use of an antibody to c-Met, the HGF receptor, linked to Gd-DTPA-albumin, reported to enhance a mouse model of glioma for up to 3 hours after administration. All other examples use Gd chelates with hundreds to thousands of Gd ions per targeting molecule. The PTPμ-directed CA, SBK2-Lys-(Gd-DOTA), with its single Gd chelate demonstrates a significant advance in the field of molecular MR agents and tumor detection. Furthermore, the specific detection of tumors will allow for improved evaluation of therapeutic efficacy.

These data clearly demonstrate that single Gd-containing PTPμ molecular agents, when combined with dynamic quantitative MR imaging methods, can specifically bind to, be retained by, and enhance recognition of tumors in vivo over longer periods of time. Sustained tumor enhancement is likely to have a significant clinical benefit as it permits imaging to occur over a longer period of time. Analysis the slope of the line for the normalized T₁ values (clearance rate) during discrete time segments as shown in FIG. 2 illustrates that differences between non-specific and specific agents can be determined within the first 10 minutes after agent administration. Another possibility, analogous to procedures used for PET scans, includes allowing an unbound specific agent like SBK2 to clear out of an area of interest prior to performing the MRI. The conventional chemical approach used to generate these contrast agents also will be useful in the development of other molecular MR agents. In addition, these simplified molecular contrast agents will lead to improved tumor detection and monitoring of therapeutic efficacy in the clinic.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. An agent for use in detecting, monitoring, and/or imaging cancer cells and/or cancer cell metastasis, migration, dispersal, and/or invasion, and/or for treating cancer in a subject, comprising:
a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment; and
a single chelating agent and metal radiolabel that are directly or indirectly linked to the targeting peptide.

2. The agent of claim 1, wherein the targeting peptide is coupled to the chelating agent and single metal radiolabel via a linking molecule.

3. (canceled)

4. (canceled)

5. The agent of claim 1, the targeting peptide comprising a polypeptide having an amino acid sequence that has at least 80% sequence identity to about 10 to about 50 consecutive amino acids of SEQ ID NO: 3.

6. The agent of claim 1, the targeting peptide comprising a polypeptide having an amino acid sequence selected from the group consisting of SEQ ID NO: 4, SEQ ID NO: 5, SEQ ID NO: 6, SEQ ID NO: 7, and SEQ ID NO: 8.

7. (canceled)

8. The agent of claim 1, the chelating agent comprising dodecanetetraacetic acid (DOTA).

9. The agent of claim 1, wherein the metal radiolabel is selected from the group consisting of a gadolinium ion or a gallium ion.

10. The agent of claim 2, wherein the linking molecule is not a contiguous portion of either the targeting peptide or chelating agent and which covalently joins an amino acid of the targeting peptide to a carboxyl group of the chelating agent.

11. The agent of claim 1, comprising the formula (I): 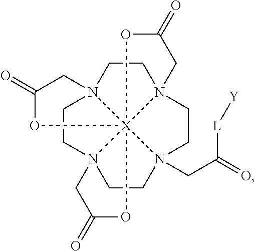
wherein X is selected from Ga or Gd metal ion,
L is a linking molecule, and
Y is a polypeptide having an amino acid sequence selected from the group consisting of SEQ ID NO: 4, SEQ ID NO: 5, SEQ ID NO: 6, SEQ ID NO: 7, and SEQ ID NO: 8.

12. The agent of claim 11, the linking molecule comprising a lysine residue.

13. (canceled)

14. A method of detecting cancer cells and/or cancer cell metastasis, migration, dispersal, and/or invasion, the method comprising:
administering to a subject an amount of a molecular probe, the molecular probe comprising a targeting peptide, wherein the targeting peptide is a polypeptide that specifically binds to and/or complexes with a proteolytically cleaved extracellular fragment of an immunoglobulin (Ig) superfamily cell adhesion molecule that is expressed by a cancer cell or another cell in the cancer cell microenvironment, and a detectable moiety that is directly or indirectly linked to the targeting peptide, the detectable moiety including a single chelating agent and metal radiolabel; and
detecting molecular probes bound to and/or complexed with the cancer cells to determine the location and/or distribution of the cancer cells in the subject.

15. The method of claim 14, wherein the targeting peptide is coupled to the detectable moiety via a linking molecule.

16. (canceled)

17. (canceled)

18. The method of claim 14, the targeting peptide comprising a polypeptide having an amino acid sequence that has at 80% sequence identity to about 10 to about 50 consecutive amino acids of SEQ ID NO: 3.

19. The method of claim 14, the targeting peptide comprising a polypeptide having an amino acid sequence selected from the group consisting of SEQ ID NO: 4, SEQ ID NO: 5, SEQ ID NO: 6, SEQ ID NO: 7, and SEQ ID NO: 8.

20. (canceled)

21. The method of claim 14, the chelating agent comprising dodecanetetraacetic acid (DOTA).

22. The method of claim 14, wherein the metal radiolabel is selected from the group consisting of a gadolinium ion or a gallium ion.

23. The method of claim 14, wherein the linking molecule is not a contiguous portion of either the targeting peptide or chelating agent and which covalently joins an amino acid of the targeting peptide to a carboxyl group of the chelating agent.

24. The method of claim 14, wherein the molecular probe comprising the formula (I): 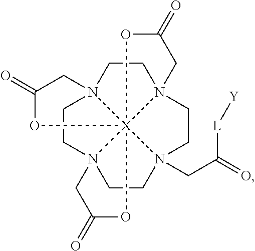
wherein X is selected from Ga or Gd metal ion,
L is a linking molecule, and
Y is a polypeptide having an amino acid sequence selected from the group consisting of SEQ ID NO: 4, SEQ ID NO: 5, SEQ ID NO: 6, SEQ ID NO: 7, and SEQ ID NO: 8.

25. The method of claim 24, the linking molecule comprising a lysine residue.

26. The method of claim 14, the detectable moiety being detected by at least one of magnetic resonance imaging positron emission tomography (PET) imaging, computer tomography (CT) imaging, gamma imaging, near infrared imaging, or fluorescent imaging.

27. The method of claim 14, the cancer cell comprising at least one of a glioma, lung cancer, melanoma, breast cancer, or prostate cancer cell.

28. The method of claim 14, the amount of the molecular probe being administered systemically to the subject.

29.-35. (canceled)
